(19) United States
(12) Patent Application Publication
(10) Pub. No.: US 2022/0378604 A1
(43) Pub. Date: Dec. 1, 2022

(54) COLD AND HEAT COSMETIC INSTRUMENT AND COLD AND HEAT COSMETIC MECHANISM COMPRISING THE SAME

(71) Applicant: Puzhen Life Co., Ltd., Shatin (HK)

(72) Inventor: Andy LEE, Shatin (HK)

(73) Assignee: Puzhen Life Co., Ltd., Shatin (HK)

(21) Appl. No.: 17/395,619

(22) Filed: Aug. 6, 2021

(30) Foreign Application Priority Data
May 28, 2021 (CN) 202121182960.6

(51) Int. Cl. A61F 7/00; H02J 7/00; H02J 50/00; H02J 50/10; A61H 23/02

(52) U.S. Cl. CPC A61F 7/007; H02J 7/0044; H02J 50/005; H02J 50/10; A61H 23/02; A61H 2201/169; A61H 2201/0207; A61H 2201/0214; A61F 2007/0093; A61F 2007/0077

(57) ABSTRACT

A cold and heat cosmetic instrument, including: a casing, a jade massage piece, a heat sink, and a cooling and heating piece. The casing defines therein an opening, the jade massage piece is arranged at the opening, and the casing and the jade massage piece are sealed to form a first accommodation chamber. The heat sink and the cooling and heating piece are arranged within the first accommodation chamber. The cooling and heating piece is in contact with the heat sink and the jade massage piece, respectively. A cold and heat cosmetic mechanism includes: a wireless charging station and the cold and heat cosmetic instrument. A receiver device for electromagnetic induction charging is arranged within the first accommodation chamber. A wireless charging station defines therein a second accommodation chamber. A transmitter device for electromagnetic induction charging is accommodated in the second accommodation chamber and connected with a charging data cable.

### CROSS-REFERENCE TO RELATED APPLICATIONS

This application claims priority to Chinese Patent Application No. 202121182960.6 filed on May 28, 2021, the content of which is incorporated herein by reference.

### BACKGROUND

### Technical Field

The present application relates to the technical field of cosmetic instruments, and more particularly to a cold and heat cosmetic instrument and a cold and heat cosmetic mechanism comprising the same.

### Description of Related Art

With the accelerated pace of modern life and the influence of factors including the air and the environment, the condition of human skin is not optimistic, and people are paying more and more attention to the care of skin, especially facial skin. As a result, various facial beauty instruments appear in the market, including beauty instruments having cold and hot compress functions. The existing cold and heat cosmetic instrument utilizes a cooling and heating piece to achieve the cold and hot compresses, and a fan is often arranged in the cold and heat cosmetic instrument to dissipate heat and increase the switching speed between the cold and hot compresses.

As being used in various usage scenarios, the cold and heat cosmetic instrument needs to be waterproof to adapt to different usage scenarios, however, the cold and heat cosmetic instrument needs to have a good sealing performance for achieving the waterproof function, therefore, a fan is not adapted to be used in the cold and heat cosmetic instrument in order to increase the switching speed of the cold and hot compresses of the cold and heat cosmetic instrument.

### SUMMARY

It is an objective of the present application to provide a the cold and heat cosmetic instrument, in order to solve the technical problem that the existing cold and heat cosmetic instrument cannot quickly switch between cold and hot compress.

To achieve the above objective, the present application adopts the following technical solutions:

A cold and heat cosmetic instrument is provided. The cold and heat cosmetic instrument comprises: a casing, a jade massage piece, a heat sink, and a cooling and heating piece. The casing defines therein an opening, the jade massage piece is arranged at the opening, and the casing and the jade massage piece are sealed together to form a first accommodation chamber. The heat sink and the cooling and heating piece are arranged within the first accommodation chamber. The cooling and heating piece is in contact with the heat sink and the jade massage piece, respectively.

In some embodiments, the cold and heat cosmetic instrument further comprises a heat transfer piece arranged within the first accommodation chamber. A first surface of the heat transfer piece is in contact with a surface of the cooling and heating piece far away from the heat sink. A second surface of the heat transfer piece is in contact with an inner wall of the jade massage piece.

In some embodiments, the cold and heat cosmetic instrument further comprises a spring arranged in the first accommodation chamber; and the spring is adapted to enable the heat sink to lean against the cooling and heating piece.

In some embodiments, the heat sink defines therein a first recess, the spring is arranged inside the first recess, and two ends of the spring are in abut connection with an inner wall of the casing and a bottom surface of the first recess, respectively.

In some embodiments, the cold and heat cosmetic instrument further comprises a vibration motor, and the vibration motor is arranged within the first accommodation chamber and adapted to generate vibration of the casing and the jade massage piece.

In some embodiments, the cold and heat cosmetic instrument further comprises a control circuit board and a power supply which are arranged within the first accommodation chamber. The control circuit board is in electric connection with the cooling and heating piece. The power supply is adapted to supply an electric power to the control circuit board and the cooling and heating piece.

The present application further provides a cold and heat cosmetic mechanism. The cold and heat cosmetic mechanism comprises: a wireless charging station and the above-described cold and heat cosmetic instrument. A receiver device for electromagnetic induction charging is arranged within the first accommodation chamber. A wireless charging station defines therein a second accommodation chamber. A transmitter device for electromagnetic induction charging is arranged within the second accommodation chamber. A charging data cable is arranged at the wireless charging station. The transmitter device for electromagnetic induction charging is in connection with the charging data cable.

In some embodiments, the wireless charging station defines therein a second recess having a shape fitted with a shape of the cold and heat cosmetic instrument. The second recess is adapted to receive the cold and heat cosmetic instrument.

In some embodiments, a heat dissipating fan is arranged inside the second accommodation chamber, and the wireless charging station defines therein an air inlet and an air outlet which are in communication with the second accommodation chamber, respectively.

In some embodiments, a surface of the wireless charging station is provided with an electroplated plastic layer.

Compared with the conventional cold and heat cosmetic instrument, advantages of the cold and heat cosmetic instrument provided by the present application are summarized as follows: the casing and the jade massage piece are sealed together to form the first accommodation chamber, so the cold and heat cosmetic instrument is waterproof, making the cold and heat cosmetic instrument more applicable and more extensively used. Because the cooling and heating piece is in contact with the heat sink and the jade massage piece, respectively, the heat sink is used to quickly transfer the energy from the cooling and heating piece via the surface thereof in contact with the heat sink. According to the law of conservation of energy, the energy of the surface of cooling and heating piece in contact with the jade massage piece also changes rapidly, such that the cold and heat cosmetic instrument 10 is adapted to quickly switch between cold and hot compresses.

### BRIEF DESCRIPTION OF THE DRAWINGS

In order to more clearly illustrate the technical solutions in the embodiments of the present application, the drawings that need to be used in the description of the embodiments or the prior art will be briefly described hereinbelow. Obviously, the accompanying drawings in the following description are only some embodiments of the present application. For those skilled in the art, other drawings can be obtained based on these drawings without creative work.

FIG. 1 is a first structural view of a cold and heat cosmetic instrument provided by an embodiment of the present application;

FIG. 2 is a first exploded view of a cold and heat cosmetic instrument provided by an embodiment of the present application;

FIG. 3 is a second exploded view of a cold and heat cosmetic instrument provided by an embodiment of the present application;

FIG. 4 is a first structural view of a wireless charging station for a cold and heat cosmetic instrument provided by an embodiment of the present application;

FIG. 5 is a second structural view of a wireless charging station for a cold and heat cosmetic instrument provided by an embodiment of the present application;

FIG. 6 is a structural view of a cold and heat cosmetic mechanism provided by an embodiment of the present application; and

FIG. 7 is an internal structural view of a wireless charging station of a cold and heat cosmetic mechanism provided by an embodiment of the present application.

In the Drawings, the Following Reference Numerals are Adopted:

10: Cold and heat cosmetic instrument; 1: Casing; 11: Opening; 12: Upper casing; 13: Lower casing; 2: Jade massage piece; 3: First accommodation chamber; 41: Heat sink; 411: First recess; 42: Cooling and heating piece; 43: Heat transfer piece; 431: First surface; 432: Second surface; 44: Spring; 45: Vibration motor; 46: Control circuit board; 47: Power supply; 20: Wireless charging station; 201: Second accommodation chamber; 202: Transmitter device for electromagnetic induction charging; 203: Charging data cable; 204: Second recess; 205: Heat dissipating fan; 206: Air inlet; and 207: Air outlet.

### DETAILED DESCRIPTION OF THE EMBODIMENTS

In order to make the purposes, technical solutions, and advantages of the present application clearer and more understandable, the present application will be further described in detail hereinafter with reference to the accompanying drawings and embodiments. It should be understood that the embodiments described herein are only intended to illustrate but not to limit the present application.

It should be understood that terms “length”, “width”, “upper”, “lower”, “front”, “rear”, “left”, “right”, “vertical”, “horizontal”, “top”, “bottom”, “inside”, “outside” and the like indicating orientation or positional relationship are based on the orientation or the positional relationship shown in the drawings, and are merely for facilitating and simplifying the description of the present application, rather than indicating or implying that a device or component must have a particular orientation, or be configured or operated in a particular orientation, and thus should not be construed as limiting the application.

It should be understand that terms like “first” and “second” are only used for the purpose of description, and will in no way be interpreted as indication or hint of relative importance or implicitly indicate the number of the referred technical features. Thus, the features prefixed by “first” and “second” will explicitly or implicitly represent that one or more of the referred technical features are included. In the description of the present application, “multiple”/“a plurality of” refers to the number of two or more than two, unless otherwise clearly and specifically defined.

In the present application, unless otherwise specifically stipulated and defined, terms like “install”, “connect”, “couple”, “fix” should be construed broadly, for example, they may indicate a fixed connection, a detachable connection, or an integral as a whole; may be a mechanical connection, or an electrical connection; may be in direct connection, or indirect connection via an intermediate, and may also reflect internal communication of two elements or interactions between two elements. For those skilled in the art, the specific meanings of the above terms in the present application can be understood according to specific conditions.

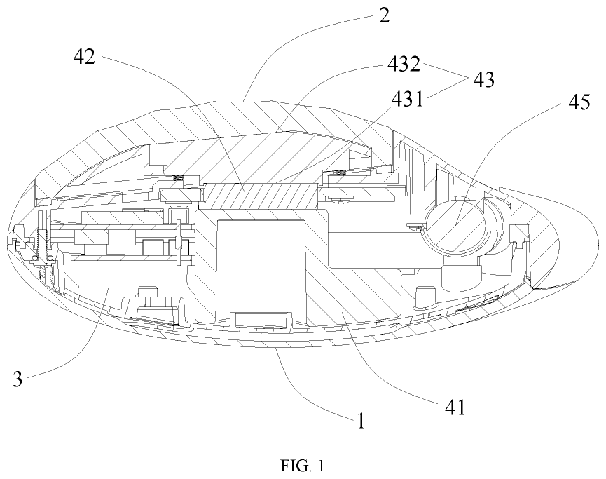

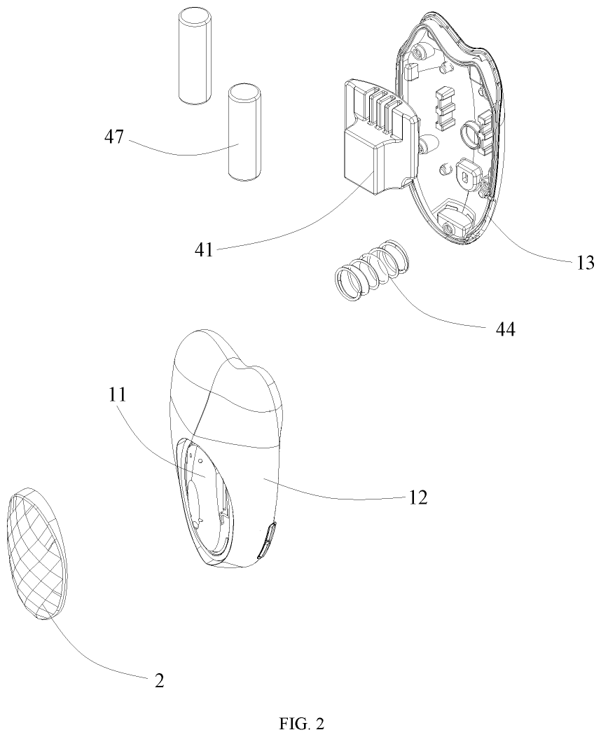

As shown in FIGS. 1-2, a cold and heat cosmetic instrument 10 provided by the present application is illustrated. The cold and heat cosmetic instrument 10 comprises: a casing 1, a jade massage piece 2, a heat sink 41, and a cooling and heating piece 42. The casing 1 defines therein an opening 11, the jade massage piece 2 is arranged at the opening 11, and the casing 1 and the jade massage piece 2 are sealed together to form a first accommodation chamber 3. The heat sink 41 and the cooling and heating piece 42 are arranged within the first accommodation chamber 3. The cooling and heating piece 42 is in contact with the heat sink 41 and the jade massage piece 2, respectively. In some embodiments, the casing 1 comprises an upper casing 12 and a lower casing 13. The opening 11 is defined in the upper casing 12, the jade is arranged at the opening 11 of the upper casing 12. The upper casing 12 is an upper casing 12 made of an electroplated aluminum alloy, and the lower casing 13 is a lower casing 13 made of an electroplated plastic. The configuration of the upper casing 12 as the upper casing 12 made of the electroplated aluminum alloy is conducive to the transfer of the vibration generated by the vibration motor 45. The configuration of the lower casing 13 as the lower casing 13 made of the electroplated plastic is conducive to alleviating the weight of the cold and heat cosmetic instrument 10, as well as improving the performance of the cold and heat cosmetic instrument 10 in aspects including electrical property, thermal property, and corrosion resistance. In some embodiments, the cooling and heating piece 42 adopts a semiconductor chilling plate, also called a thermoelectric chilling plate, which utilizes the Seebeck effect, Peltier effect, and Thomson effect of semiconductor materials. When the direct current passes through the semiconductor chilling plate, the two length-width surfaces of the semiconductor chilling plate can realize the heat absorption and release, respectively, of which, one of the length-width surfaces of the semiconductor chilling plate that is adapted to absorb heat is called a cold surface, and the other one of length-width surfaces of the semiconductor chilling plate that is adapted to release heat is called a hot surface, such that the semiconductor chilling plate is capable of cooling, with a temperature difference between the cold surface and the hot surface capable of reaching about 60° C.; in addition, the cold surface and the hot surface can also switch according to practical needs. In specific implementation, when the cooling and heating piece 42 is working, a contact surface at one side thereof is configured to heat, and another contact surface at the opposite side thereof is configured to cool. Two sides of the cooling and heating piece 42 are respectively in contact with the heat sink 41 and the jade massage piece, whereby the heat sink 41 and the jade massage piece 2 have different hot and cold effects. When the jade massage piece 2 is used to apply a hot compress to a human face, a surface of the cooling and heating piece 42 in contact with the heat sink 41 serves as the cold surface, and the heat sink 41 can quickly transfer the cooling from the cooling and heating piece 42 via the cold surface thereof. According to the law of conservation of energy, the temperature of the hot surface will increase. Since the hot surface is in contact with the jade massage piece 2, the jade massage piece 2 can apply the heat compress to the human face. When the jade massage piece 2 is used to apply a cold compress to the human face, a surface of the cooling and heating piece 42 in contact with the heat sink 41 serves as the hot surface, and the heat sink 41 can quickly transfer the heating from the cooling and heating piece 42 via the hot surface thereof. According to the law of conservation of energy, the temperature of the cold surface will decrease. Since the cold surface is in contact with the jade massage piece 2, the jade massage piece 2 can apply the cold compresses to the human face. Due to the function of the heat sink 41, the side of the cooling and heating piece 42 in contact with the heat sink 41 can accelerate the transfer of energy, so that the side of the cooling and heating piece 42 in contact with the jade massage piece 2 can quickly realize cooling or heating, which can quickly switch between hot compress and cold compress.

Compared with the conventional cold and heat cosmetic instrument, advantages of the cold and heat cosmetic instrument 10 provided by the present application are summarized as follows: the casing 1 and the jade massage piece 2 are sealed together to form the first accommodation chamber 3, so the cold and heat cosmetic instrument 10 is waterproof, making the cold and heat cosmetic instrument 10 more applicable and more extensively used. Because the cooling and heating piece 42 is in contact with the heat sink 41 and the jade massage piece 2, respectively, the heat sink 41 is used to quickly transfer the energy from the cooling and heating piece 42 via the surface thereof in contact with the heat sink 41. According to the law of conservation of energy, the energy of the surface of cooling and heating piece 42 in contact with the jade massage piece 2 also changes rapidly, such that the cold and heat cosmetic instrument 10 is adapted to quickly switch between cold and hot compresses.

Further, as shown in FIG. 1, as a specific embodiment of the cold and heat cosmetic instrument 10 provided by the present application, the cold and heat cosmetic instrument 10 further comprises a heat transfer piece 43 arranged within the first accommodation chamber 3. A first surface 431 of the heat transfer piece 43 is in contact with a surface of the cooling and heating piece 42 far away from the heat sink 41. A second surface 432 of the heat transfer piece 43 is in contact with an inner wall of the jade massage piece. In specific implementation, the heat transfer piece 43 can achieve heating conduction and cooling conduction, and can be made of gold, silver, copper, ferrum, and tin, as well as other materials, as long as such material can achieve heating conduction and cooling conduction, and those skilled in the art can choose a proper material according to practical needs. Due to the irregular shape of the jade massage piece 2, the cooling and heating piece 42 cannot be fully fitted with the jade massage piece 2 when being in contact with the jade massage piece 2, which adversely affects the energy transfer effect, results in uneven surface temperature of jade massage piece 2, and affects the effect of hot and cold compresses. Therefore, the arrangement of the heat transfer piece 43 between the jade massage piece 2 and the cooling and heating piece 42 enables the first surface 431 thereof to be in complete contact with the cooling and heating piece 42 and enables the second surface 432 thereof to be in contact with the inner wall of the jade massage piece 2. In this way, the heat transfer piece 43 can better transfer the energy from the cooling and heating piece 42 to the jade massage piece 2, and in the meanwhile ensure even energy distribution on the surface of the jade massage piece 2, thereby realizing better effect of hot and cold compresses on the human face.

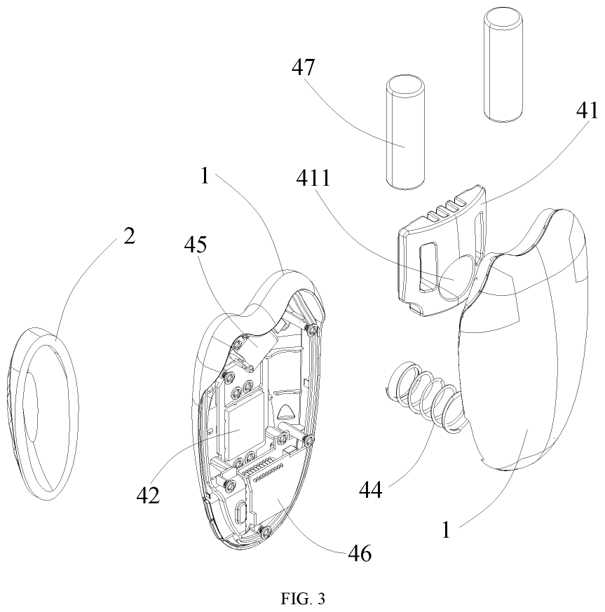

Further, as shown in FIGS. 2-3, as a specific embodiment of the cold and heat cosmetic instrument 10 provided by the present application, the cold and heat cosmetic instrument 10 further comprises: a spring 44 arranged in the first accommodation chamber 3; and the spring 44 is adapted to enable the heat sink 41 to abut against the cooling and heating piece 42. In use of the cold and heat cosmetic instrument 10, the heat sink 41 may not be in tight contact with the cooling and heating piece 42, which would affect the cooling or heating effect of the cold and heat cosmetic instrument 10. The arrangement of the spring 44 can make the heat sink 41 abut against the cooling and heating piece 42, thereby ensuring the cooling and heating effect of the cold and heat cosmetic instrument 10.

Further, as shown in FIGS. 2-3, as a specific embodiment of the cold and heat cosmetic instrument 10 provided by the present application, the heat sink 41 defines therein a first recess 411, the spring 44 is arranged inside the first recess 411, and two ends of the spring 44 are in abut connection with an inner wall of the casing 1 and a bottom surface of the first recess 411, respectively. In specific implementation, as the heat sink 41 defines therein a first recess 411, the spring 44 is arranged inside the first recess 411, and two ends of the spring 44 are in abut connection with an inner wall of the casing 1 and a bottom surface of the first recess 411, respectively, therefore, under the action of the spring 44, the heat sink 41 can make the heat sink 41, the cooling and heating piece 42, the heat transfer piece 43, and the jade massage piece 2 in tighter contact, such that the cooling and heating effect reaches the optimal state, the reduction in contact area between the cooling and heating piece 42 and the heat sink 41 caused by changes in processing dimensions and assembling methods is prevented, which would otherwise obviously lowering the heating and cooling temperature and speed.

Further, as shown in FIG. 3, as a specific embodiment of the cold and heat cosmetic instrument 10 provided by the present application, the cold and heat cosmetic instrument 10 further comprises a vibration motor 45 which is arranged within the first accommodation chamber 3 and adapted to generate vibration of the casing 1 and the jade massage piece 2. Due to the arrangement of the vibration motor 45, the cold and heat cosmetic instrument 10 can perform vibration massage on the human face during hot and cold compresses, thereby increasing the application range of the cold and heat cosmetic instrument 10.

Further, as shown in FIG. 3, as a specific embodiment of the cold and heat cosmetic instrument 10 provided by the present application, the cold and heat cosmetic instrument 10 further comprises a control circuit board 46 and a power supply 47 which are arranged within the first accommodation chamber 3. The control circuit board 46 is in electric connection with the cooling and heating piece 42; and the power supply 47 is adapted to supply an electric power to the control circuit board 46 and the cooling and heating piece 42. The control circuit board 46 is capable of realizing the control of the cooling and heating piece 42, and the power supply 47 can provide power for the control circuit board 46 and the cooling and heating piece 42.

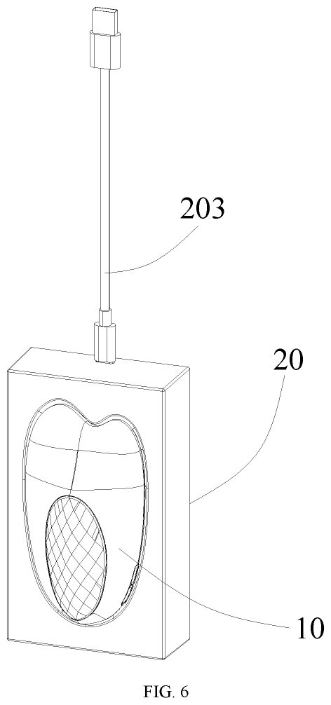

The present application further provides a cold and heat cosmetic mechanism. As shown in FIG. 6, the cold and heat cosmetic mechanism comprises: a wireless charging station 20 and the cold and heat cosmetic instrument 10 as described in the above. A receiver device for electromagnetic induction charging (not shown in the figures) is arranged within the first accommodation chamber 3. A wireless charging station 20 defines therein a second accommodation chamber 201. A transmitter device 202 for electromagnetic induction charging is arranged within the second accommodation chamber 201. A charging data cable 203 is arranged at the wireless charging station 20. The transmitter device 202 for electromagnetic induction charging is in connection with the charging data cable 203. According to the principle of electromagnetic induction, electromagnetic induction wireless charging is equipped with a closed coil at each of the energy transmitter terminal and the energy receiver terminal, respectively. The current passes through the transmitter coil to generate an alternating magnetic field, and imposes an induced electromotive force on the nearby receiver coil, so as to generate an electric current to realize wireless power transmission. In specific implementation, when the cold and heat cosmetic instrument 10 has insufficient energy and needs to be charged, the cold and heat cosmetic instrument 10 can be placed on the wireless charging station 20, and the charging data cable 203 of the wireless charging station 20 is then connected to an external power supply 47, such that the current transmitted to the wireless charging station 20 through the charging data cable 203 can be wirelessly transmitted to the receiver device for electromagnetic induction charging through the transmitter device 202 for electromagnetic induction charging in a manner of electromagnetic induction, thereby realizing the charging of the cold and heat cosmetic instrument 10.

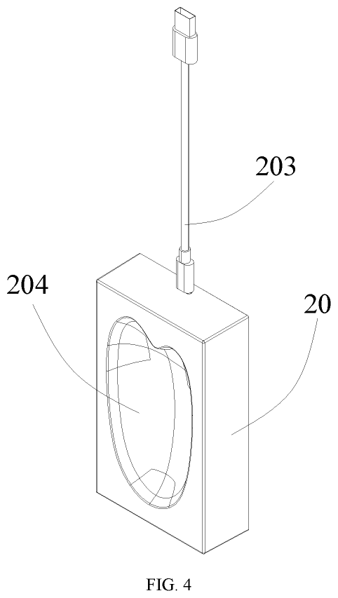

Further, as shown in FIG. 4, as a specific embodiment of the cold and heat cosmetic mechanism provided by the present application, the wireless charging station 20 defines therein a second recess 204 having a shape fitted with a shape of the cold and heat cosmetic instrument 10. The second recess 204 is adapted to receive the cold and heat cosmetic instrument 10. By defining the second recess 204 in the wireless charging station 20 to have a shape of the second recess 204 fitted with that of the cold and heat cosmetic instrument 10, when the cold and heat cosmetic instrument 10 is placed in the second recess 204, it is ensured that the cold and heat cosmetic instrument 10 can be charged steadily during the charging process. In specific implementation, the cold and heat cosmetic instrument 10 can be partially placed in the second recess 204 or completely placed in the second recess 204, as long as the stability of the cold and heat cosmetic instrument 10 in the charging process is ensured, and those skilled in the art can select proper arrangements according to practical needs.

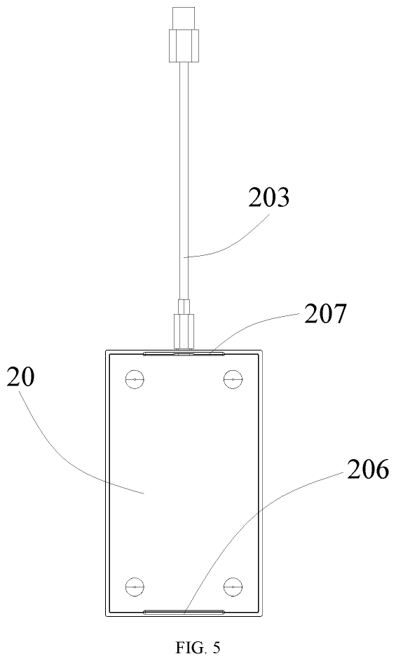

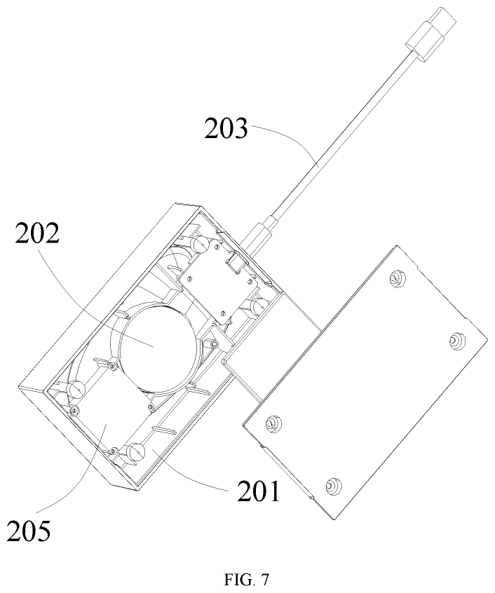

Further, as shown in FIGS. 5 and 7, as a specific embodiment of the cold and heat cosmetic mechanism provided by the present application, a heat dissipating fan 205 is arranged inside the second accommodation chamber 201, and the wireless charging station 20 defines therein an air inlet 206 and an air outlet 207 which are in communication with the second accommodation chamber 201, respectively. By the arrangement of the heat dissipating fan 205 in the second accommodation chamber 201 of the wireless charging station 20, the wireless charging station 20 can be dissipated by the heat dissipating fan 205 when charging the cold and heat cosmetic instrument 10, thereby preventing the temperature of the wireless charging station 20 from being too high.

Further, as a specific embodiment of the cold and heat cosmetic mechanism provided by the present application, a surface of the wireless charging station 20 is provided with an electroplated plastic layer. The arrangement of the electroplated plastic layer on the surface of the wireless charging station 20 can improve the performance of the cold and heat cosmetic instrument 10 in aspects including electrical property, thermal property, and corrosion resistance, and extending the service life thereof.

The above are only preferred embodiments of the present application and are not intended to limit the present application. Any modification, equivalent replacement, and improvement made within the spirit and principle of the present application shall be included within the protection scope of the present application.

What is claimed is:

1. A cold and heat cosmetic instrument, comprising: a casing, a jade massage piece, a heat sink, and a cooling and heating piece;
wherein
the casing defines therein an opening, the jade massage piece is arranged at the opening, and the casing and the jade massage piece are sealed together to form a first accommodation chamber;
the heat sink and the cooling and heating piece are arranged within the first accommodation chamber; and
the cooling and heating piece is in contact with the heat sink and the jade massage piece, respectively.

2. The cold and heat cosmetic instrument of claim 1, wherein
the cold and heat cosmetic instrument further comprises a heat transfer piece arranged within the first accommodation chamber;
a first surface of the heat transfer piece is in contact with a surface of the cooling and heating piece far away from the heat sink; and
a second surface of the heat transfer piece is in contact with an inner wall of the jade massage piece.

3. The cold and heat cosmetic instrument of claim 1, wherein
the cold and heat cosmetic instrument further comprises a spring arranged in the first accommodation chamber; and the spring is adapted to enable the heat sink to lean against the cooling and heating piece.

4. The cold and heat cosmetic instrument of claim 3, wherein
the heat sink defines therein a first recess, the spring is arranged inside the first recess, and two ends of the spring are in abut connection with an inner wall of the casing and a bottom surface of the first recess, respectively.

5. The cold and heat cosmetic instrument of claim 1, wherein
the cold and heat cosmetic instrument further comprises a vibration motor, and the vibration motor is arranged within the first accommodation chamber and adapted to generate vibration of the casing and the jade massage piece.

6. The cold and heat cosmetic instrument of claim 1, wherein
the cold and heat cosmetic instrument further comprises a control circuit board and a power supply which are arranged within the first accommodation chamber;
the control circuit board is in electric connection with the cooling and heating piece; and
the power supply is adapted to supply an electric power to the control circuit board and the cooling and heating piece.

7. A cold and heat cosmetic mechanism, comprising: a wireless charging station and the cold and heat cosmetic instrument according to claim 1;
wherein
a receiver device for electromagnetic induction charging is arranged within the first accommodation chamber;
a wireless charging station defines therein a second accommodation chamber;
a transmitter device for electromagnetic induction charging is arranged within the second accommodation chamber;
a charging data cable is arranged at the wireless charging station; and
the transmitter device for electromagnetic induction charging is in connection with the charging data cable.

8. The cold and heat cosmetic mechanism of claim 7, wherein
the wireless charging station defines therein a second recess having a shape fitted with a shape of the cold and heat cosmetic instrument; and
the second recess is adapted to receive the cold and heat cosmetic instrument.

9. The cold and heat cosmetic mechanism of claim 7, wherein
a heat dissipating fan is arranged inside the second accommodation chamber, and the wireless charging station defines therein an air inlet and an air outlet which are in communication with the second accommodation chamber, respectively.

10. The cold and heat cosmetic mechanism of claim 7, wherein a surface of the wireless charging station is provided with an electroplated plastic layer.

11. The cold and heat cosmetic mechanism of claim 7, wherein
the cold and heat cosmetic instrument further comprises a heat transfer piece arranged within the first accommodation chamber;
a first surface of the heat transfer piece is in contact with a surface of the cooling and heating piece far away from the heat sink; and
a second surface of the heat transfer piece is in contact with an inner wall of the jade massage piece.

12. The cold and heat cosmetic mechanism of claim 7, wherein
the cold and heat cosmetic instrument further comprises a spring arranged in the first accommodation chamber; and the spring is adapted to enable the heat sink to lean against the cooling and heating piece.

13. The cold and heat cosmetic mechanism of claim 12, wherein
the heat sink defines therein a first recess, the spring is arranged inside the first recess, and two ends of the spring are in abut connection with an inner wall of the casing and a bottom surface of the first recess, respectively.

14. The cold and heat cosmetic mechanism of claim 7, wherein
the cold and heat cosmetic instrument further comprises a vibration motor, and the vibration motor is arranged within the first accommodation chamber and adapted to generate vibration of the casing and the jade massage piece.

15. The cold and heat cosmetic mechanism of claim 7, wherein
the cold and heat cosmetic instrument further comprises a control circuit board and a power supply which are arranged within the first accommodation chamber;
the control circuit board is in electric connection with the cooling and heating piece; and
the power supply is adapted to supply an electric power to the control circuit board and the cooling and heating piece.
